(19) United States
(12) Patent Application Publication
(10) Pub. No.: US 2026/0224776 A1
(43) Pub. Date: Aug. 6, 2026

(54) Biphasic Glucomannan Scaffolds and Methods of Use

(71) Applicant: Anemorix, LLC, Rancho Cordova, CA (US)

(72) Inventors: C. Chang I. Lee, Oceanside, CA (US); Justin Judd, Placerville, CA (US)

(73) Assignee: Anemorix, LLC, Rancho Cordova, CA (US)

(21) Appl. No.: 19/153,310

(22) PCT Filed: Feb. 2, 2024
(86) PCT No.: PCT/US2024/014165

§ 371 Date: Aug. 1, 2025

Related U.S. Application Data
(60) Provisional application No. 63/443,300, filed on Feb. 3, 2023.

(51) Int. Cl. A61L 27/20; A61L 27/54; A61L 27/56

(52) U.S. Cl. CPC A61L 27/20; A61L 27/54; A61L 27/56; A61L 2300/406; A61L 2300/414; A61L 2300/45; A61L 2400/06; A61L 2430/02; A61L 2430/12; A61L 2430/38

(57) ABSTRACT

The biphasic scaffolds of the present invention comprise both a solid porous component and a flowable component that is disposed within the pores of the solid component.

### CROSS REFERENCE TO RELATED APPLICATIONS

This application claims priority to U.S. Provisional Appl. No. 63/443,300 filed 3 Feb. 2023, which is incorporated by reference herein in its entirety.

### BACKGROUND OF INVENTION

Carbohydrate-based scaffolds have been used as carrier vehicles for osteoinductive factors such as recombinant human BMP-2. rhBMP-2 has been used for off-label anterior cervical discectomy and fusion (ACDF) procedures, but at concentrations far exceeding naturally occurring BMP-2 levels to compensate for leakage beyond implant site into adjacent tissues. Such leakage and high dose amounts are associated with adverse events. Thus, a carrier effective to localize BMP-2 at the implant site and to lower the effective dose is desirable.

### BRIEF SUMMARY OF THE INVENTION

The present invention describes biphasic scaffolds and methods of preparing and using them.

In one embodiment, the present invention provides biphasic scaffolds that include a solid porous component (shaped integral matrix, chips, granules, etc.) and a flowable component (e.g., liquid, gel, etc.).

In another embodiment, the biphasic scaffolds described herein can be used in tissue engineering applications (experimental or therapeutic). They can be used as a carrier to deliver growth factors and/or other pharmaceutically active agents.

### BRIEF DESCRIPTION OF THE DRAWINGS

FIGS. 1A-1D show macroscopic characteristics of representative osteoinductive implants at 4 weeks post implant. FIGS. 1A and 1C show biphasic hyper-crosslinked carbohydrate polymer (HCCP) with 75 μg rhBMP-2. FIGS. 1B and 1D show absorbable collagen sponge (ACS) with 75 μg rhBMP-2.

FIGS. 2A-2L shows representative H&E images of osteoinductive implants at 4 weeks post implant (dissection scope). FIGS. 2A-2F show biphasic HCCP with 75 μg rhBMP-2. FIGS. 2G-2L show ACS with 75 μg rhBMP-2. The scale bar ticks measure 1 millimeter.

FIGS. 2M-2P show representative H&E images used to quantify percent bone (Nikon, 4×). FIGS. 2M and 2O show biphasic HCCP with 75 μl rhBMP-2. FIGS. 2N and 2P show ACS with 75 μl rhBMP-2.

FIGS. 3A-3D show representative osteoinductive implants stained with Alcian blue. FIGS. 3A and 3C show biphasic HCCP with 75 μg rhBMP-2. FIGS. 3B and 3D show ACS with 75 μl rhBMP-2.

FIG. 4A-F shows comparison of bone and cartilage induction in osteogenic implants with BMP2. Bone and cartilage induction was compared in Biphasic HCCP and INFUSE™ ACS osteogenic implants with rhBMP-2 by histomorphological methods. FIG. 4A is a representative example of counting method: A 10×10 grid was superimposed and point counts collected to evaluate the percentage of each section composed of mature bone or cartilaginous tissue. The percentage of mature bone (FIG. 4B) or cartilaginous tissue (FIG. 4C) to total tissue was similar in biphasic HCCP and INFUSE™ absorbable collagen sponge (ACS), p=0.18. The total tissue section area was greater in the biphasic HCCP implants than in the INFUSE™ ACS implants (FIG. 4D, ***p<0.001). The total mature bone area present in biphasic HCCP implants was increased 3.8-fold over the bone area present in INFUSE™ ACS implants (FIG. 4E, **p<0.01). Total cartilaginous area in biphasic HCCP implants was increased 4.6-fold over INFUSE™ ACS implants (FIG. 4F, ***p<0.001).

### DETAILED DESCRIPTION OF THE INVENTION

### I. Definitions

As used herein, the term “glucomannan” refers to a naturally-derived oligosaccharide composed of an approximately 1:1.6 ratio of β-1,4-linked D-glucose to D-mannose with branches approximately every 11 residues (Alonso-Sande et al. Eur J Pharm Biopharm. 2009 72:453-462) and derivatives thereof. Glucomannan has a backbone of approximately 5-10% substituted acetyl groups that participate in hydrogen bonding and hydrophobic interactions that confer solubility. Exemplary glucomannan derivatives include, but are not limited to, water-soluble derivatives such as O-alkyl derivatives and O-carboxyalkyl derivatives, derivatives with various degrees of substitution (without limiting, for example greater than or less than 5-10% substituted acetyl groups), derivatives with various degrees of oxidation, graft copolymers (without limiting for example, acrylate and acrylamide copolymers) and salts thereof (such as quaternary ammonium salts thereof).

As used herein, the term “glucomannan gel” refers to a thermally stable, homogeneous suspension of crosslinked carbohydrate). The glucomannan gel can be formed in a variety of ways including, but not limited, by hydrolysis of the acetyl groups of glucomannan in the presence of alkali.

As used herein, “porosity” means the ratio of volume of pores to the total volume of the (solid) scaffold as measured by mercury intrusion porosimetry testing.

As used herein, “interconnectivity” refers to a network of pores that are permeable and allow the flow of matter from one pore to another. Interconnectivity can be measured by a dye permeation study.

### II. Biphasic Scaffolds

In one embodiment, the present invention describes biphasic glucomannan scaffolds. The biphasic scaffolds of the present invention comprise both a solid porous component and a flowable component (e.g., liquid or gel) that is disposed within and/or between the pores of the solid porous component.

### III. Solid Porous Component

The solid porous component of the biphasic scaffolds can comprise a glucomannan gel that has been solidified into a solid porous component. In some embodiments, the solid porous component can comprise a single integral structure, or two or more integral structures (e.g., wherein the integral structures can be fabricated into a shape or dimensions according to the intended site of use). In other embodiments, the solid porous component comprises a plurality of chips or granules, such that the biphasic scaffold as a whole is structurally supported while being moldable on site. The chips or granules are of a size that is large enough to maintain porosity, while small enough to adapt to the biological site and/or intended mode of application (e.g., injectable). The biphasic scaffold can alternatively comprise a mixture of integral pieces and plurality of chips or granules. The biphasic scaffold can also include a powder component (which is solid but not porous) to adjust the viscosity of the flowable component as described below.

In some embodiments, the solid porous component is formed by making a reaction mixture including carbohydrate mixture with at least about 50% (w/w) glucomannan, an alkaline solution, and water; and heating the reaction mixture at a temperature of about 50° C. to about 130° C. to form a glucomannan gel and/or increasing the pressure of the glucomannan gel to about 0.1 psi to 50 psi above atmospheric pressure to form a glucomannan gel.

The glucomannan gel used in the method of the present invention is a mixture of a carbohydrate mixture with at least about 50% (w/w) glucomannan and an aqueous solution. The glucomannan can be provided as glucomannan powder. In some embodiments, glucomannan powder is dissolved in water to provide a glucomannan solution containing from about 1% to about 5% w/v glucomannan in water. The glucomannan powder can be dissolved in the aqueous solution at any appropriate temperature and pressure conditions. Examples of other carbohydrates that may be included are, without limiting, alginate, chitosan, starch, plant or bacterial-based polysaccharides.

The methods and compositions herein are described with respect to glucomannan gels and glucomannan scaffolds as exemplary, but the invention can encompass gels and scaffold that are not entirely comprised of glucomannan. Other naturally derived materials (e.g., carbohydrate-or protein-based biomaterials) or synthetic materials (e.g., polymer-and ceramic-based materials) may be included or combined in the gel and/or scaffold. In the embodiments of the present invention, the scaffold comprises at least about 50% (w/w) glucomannan, in some embodiments at least 60%, 70%, 80%, 90%, 95% glucomannan (w/w).

In some embodiments, the porous solid component exhibits one or more of the following structural features:
The porosity of the solid porous component can be at least 50%, 60%, 70%, or 80%.
The interconnectivity of the porous solid component can be at least 50%, 60%, 70%, 80%, 90%, 95%, or 99% of the scaffold.
Porosity and/or interconnectivity can be presented uniformly throughout the solid porous component.
The solid porous component can exhibit a uniform pore size of about 100-500 μm.

In one embodiment, the solid porous component of the biphasic scaffold comprises the scaffold matrix as described in US Published Patent Application 2021/0123014, incorporated by reference herein in its entirety.

### IV. Flowable Component

The flowable component comprises glucomannan powder mixed with an aqueous fluid (such as sterile water for injection (SWFI), saline, blood, bone marrow, etc.). The viscosity of the flowable component can be controlled by adjusting the ratio of powder to liquid. The ratio of liquid: powder (ml:g) determines the viscosity, from stiffer putty-like formulations around 10:1 or less, to well-hydrated but cohesive formulations around 20:1. Injectables can be made with higher liquid: powder ratios. In particular, injectable formulations can employ ratios of at least 20:1, at least 40:1, at least 50:1, at least 75:1, or at least 100:1. The ratio of liquid: powder (ml:g) can be 5:1 to 50:1, 5:1 to 15:1, 10:1 to 40:1, 10:1 to 25:1, 15:1 to 25:1, 20:1 to 40:1, 20:1 to 100:1, 30:1 to 50:1, or 40:1-100:1. In certain embodiments, the ratio is about 10:1, about 15:1, about 20:1, or about 40:1.

The flowable composition component of the biphasic scaffolds can comprise a glucomannan gel as described above for the preparation of the glucomannan solid porous component, except that the gel does not undergo the solidification process. In one embodiment, the flowable component is a glucomannan gel that is the same as the glucomannan gel (before freeze-drying) used to form the solid porous component. In other embodiments, the glucomannan gel used as the flowable component is different from the glucomannan gel used to form the solid porous component.

### V. Additives and Pharmaceutically Active Agents

The biphasic scaffold can include one or more additional additives or pharmaceutically active agents specific to the targeted application. The additives and/or pharmaceutically active agents can be added to the solid porous component, the flowable component, or both. When added to the solid porous component, the additives and/or pharmaceutically active agents may be optionally fused to the backbone of the solid structure. When added to the flowable component, the additives and/or pharmaceutically active agents may be formulated as part of the powder and/or the liquid composition. In one embodiment, pharmaceutically active agents can be loaded in the biphasic scaffold by suspending them in the liquid used to hydrate the solid porous component (chips) and flowable component (powder).

Exemplary additives include, but are not limited to:
an aqueous solution, an acidic solution, or an alkaline solution;
cell adhesion promoter, chemical crosslinking, surface coating, and functional groups;
chemotactic molecules;
osteoinductive factors;
cell signaling molecules; and/or
bone constituents.

Exemplary pharmaceutically active agents include, but are not limited to:
tissue specific growth factors (e.g., granulocyte-macrophage colony stimulating factor (GM-CSF), platelet derived growth factor (PDGF), vascular endothelial growth factor (VEGF), and basic fibroblast growth factor (bFGF)) (See Barrientos S, Brem H, Stojadinovic O, Tomic-Canic M. Clinical application of growth factors and cytokines in wound healing. Wound Repair Regen. 2014 September-October; 22(5): 569-78.);
bone morphogenetic proteins (See Kissling S, Seidenstuecker M, Pilz I H, Suedkamp NP, Mayr H O, Bernstein A. “Sustained release of rhBMP-2 from microporous tricalciumphosphate using hydrogels as a carrier.” BMC Biotechnol. 2016 May 20;16(1):44.);
antibiotics (e.g., vancomycin, etc.) (See Zhang, J z., Xiao, C s., Wang, J c. et al. “Photo cross-linked biodegradable hydrogels for enhanced vancomycin loading and sustained release.” Chin J Polym Sci 31, 1697-1705 (2013); Ma T, Shang B C, Tang H, Zhou T H, Xu G L, Li H L, Chen Q H, Xu Y Q. “Nano-hydroxyapatite/chitosan/konjac glucomannan scaffolds loaded with cationic liposomal vancomycin: preparation, in vitro release and activity against Staphylococcus aureus biofilms.” J Biomater Sci Polym Ed. 2011; 22(12): 1669-81.); and/or
chemotherapeutic agents (e.g., doxorubicin, etc.) (See Eskandari S, Guerin T, Toth I, Stephenson R J. “Recent advances in self-assembled peptides: Implications for targeted drug delivery and vaccine engineering.” Adv Drug Deliv Rev. 2017 February; 110-111:169-187; Senapati, S., Mahanta, A. K., Kumar, S. et al. “Controlled drug delivery vehicles for cancer treatment and their performance.” Sig Transduct Target Ter 3, 7 (2018); Thavasyappan T et al. “Injectable hydrogels for sustained release of therapeutic agents,” Journal of Controlled Release, Volume 267 (2017) pages 57-66; Jia W. et al. “Controlled release of anticancer drug using graphene oxide as a drug-binding effector in konjac glucomannan/sodium alginate hydrogels,” Colloids and Surfaces B: Biointerfaces, Volume 113 (2014), pages 223-229.)

In one embodiment, the biphasic scaffold can be used in bone tissue engineering applications. In this context, the biphasic scaffold can preferably include one or more bone morphogenetic proteins. Bone morphogenetic proteins include, but are not limited to, BMP-1, BMP-2, BMP-3, BMP-4, BMP-5, BMP-6, BMP-7, BMP-8a, BMP-8b, BMP-9, BMP-10, BMP-11, BMP-12, BMP-13, BMP-14, BMP-15. In one embodiment, the scaffold includes BMP-2. The BMPs can be recombinant or naturally derived. The ratio of the concentration of bone morphogenetic protein solution in the scaffold is about 0.001 to 1.5 mg/mL.

The scaffold may include other osteoinductive factors such as fibroblast growth factor-2 (FGF-2), and/or platelet-derived growth factor (PDGF). In humans, BMP-2 concentrations measured in fractured bone supernatants are about 23.2 pg/mL (Glass et al. Proceedings of the National Academy of Sciences 2011 108:1585-1590). However, recombinant human BMP-2 (rhBMP-2) is delivered clinically at a dose of 1.5 mg/mL, significantly higher than the natural concentration of BMP-2 following injury. rhBMP-2 was used for off-label anterior cervical discectomy and fusion (ACDF) procedures at a concentration up to 2.5 mg/level, 3.5 times the amounts used in pilot studies (Shields et al. Spine 2006 31:542-547). This higher dosage was associated with increased complications including hematomas, neck swelling, dysphagia, and excessive edema (Shields et al. Spine 2006 31:542-547). Other adverse events traced to high doses of rhBMP-2 include swallowing difficulties and abnormal adipose tissue formation (Shields et al. Spine 2006 31:542-547). While the use of BMP-2 has increased in popularity, 85% of its usage between 2003 and 2007 was off-label (Ong et al. Spine 2010 35:1794-1800). This has resulted in BMP-2 receiving significant attention for the complications stemming from the high clinical dosages (Lykissas et al. World Journal of Orthopedics 2017 8:531-555). In addition, it is important to localize rhBMP-2 to the site of injury for osteogenic precursors to proliferate and differentiate into mature bone cells (Sandhu et al. Spine 2003 28:64-73) without leaking into adjacent tissues. However, rhBMP-2 has been shown to result in unintended side effects in the surrounding areas when its effects extend outside of the target area, resulting in ectopic bone formation in addition to the side effects listed above (Tannoury et al. The Spine Journal 2014 14:552-559, Shields et al. Spine 2006 31:542-547). A common commercial carrier for rhBMP-2 is an absorbable collagen sponge (ACS). While ACS had been shown to be an effective carrier for rhBMP-2, the fast release rate, possibly caused by collagen degradation, requires rhBMP-2 to be implanted at a high concentration to deliver an effective dose, which further increases the risk of complications (Mariner et al. Journal of Orthopaedic Research 2012 31:401-406, Winn et al. Clinical Orthopaedics and Related Research 1999 367:95-106). Thus, a carrier effective to localize rhBMP-2 at the implant site and to lower the effective dose is desirable.

In certain embodiments, the biphasic scaffold includes one or more bone constituents. Bone constituents are defined as any substance that contributes to osteogenesis, or the formation of bone. Bone constituents include, but are not limited to, organic components such as extracellular and/or bone matrices or ground bone, and inorganic components such as calcium, phosphate, potassium, magnesium, and hydroxyapatite. In one embodiment, the scaffold includes calcium, phosphate, and/or magnesium as a bone constituent. In another embodiment, the scaffold includes calcium. The bone constituent(s) can be integrally incorporated to the backbone of the solid porous component.

In some embodiments, the total concentration of bone constituents with respect to the scaffold system is in a range of about 0.1 to 95% (w/w). For example, the scaffold can be manufactured with 1-20%, 1-10%, or about 5% CaOH: glucomannan powder (w/w).

### VI. Methods of Preparing the Biphasic Scaffolds

In one embodiment, the ratio of solid phase HCCP chips: liquid: powder (g:ml:g) can be tailored to the specific requirements of the implant, with the ratio 1:30:2 yielding a moldable, cohesive, and well-hydrated implant. Accordingly, in one embodiment the ratio of solid porous to aqueous fluid to glucomannan powder (g:ml:g) is about 1:30:2.

In other embodiments, the amount of each component can be adjusted individually. For example, the following amounts of each component can be selected individually to prepare a 10 cc implant formulation, without adhering to a specific ratio of chips: liquid: powder. In one embodiment of a 10 cc formulation, the amount of solid phase HCCP chips can be 50-4000, 50-2000, 50-700, 50-200, 150-4000, 150-2000, 150-650, 200-600, 500-4000, 500-2000, 500-2000, or 2000-4000 mg. In another embodiment, the amount of solid phase HCCP chips is about 150, 200, 300, 350, 400, 500, 600, or 650 mg. In one embodiment of a 10 cc formulation, the amount of carbohydrate mixture (powdered form used to prepare the flowable portion) can be 100-6000, 100-4000, 100-1500, 100-300, 300-6000, 300-4000, 300-1500, 1250-6000, 1250-4000, or 4000-6000 mg. In another embodiment, the amount of carbohydrate mixture (powder) is about 300, 350, 600, 650, 750, 1000, or 1300 mg. In one embodiment of a 10 cc formulation, the amount of liquid (used to prepare the flowable portion) can be 6-14, 6-12, 6-8, 8-14, 8-12, or 12-14 ml. In another embodiment, the amount of liquid can be about 8, 9, 10, 11, or 12 ml. These amounts can also be scaled to yield implant formulations of smaller or larger volumes.

In one embodiment, the solid porous component is contacted with an excess of flowable composition such that the solid porous component maximally absorbs the flowable component. Other methods of using pressure (e.g., injection or spray), gravity, suction, electric, or magnetic forces to draw the flowable composition into the solid porous component would be apparent to one of ordinary skill in the art.

Not all pores need contain the flowable composition. In one embodiment, at least 50%, 60%, 70%, 75%, 80%, 90%, 95%, or 99% of the pores contain at least some flowable composition. In another embodiment, at least 50%, 60%, 70%, 75%, 80%, 90%, 95%, or 99% of the pores are filled with the flowable composition, that is, the flowable composition has replaced the air originally within the pores. The flowable composition may also be present in interstitial spaces between the pores of the solid porous component.

In some embodiments, the solid porous and flowable components are combined during manufacture into a vacuum-sealed or otherwise shelf stable packaging that maintains the structural integrity of both the solid porous and flowable components. In this case, the biphasic scaffold (e.g., the solid porous component and/or the flowable component) can comprise one or more preservatives as would be apparent to one of ordinary skill in the art.

In other embodiment, the solid porous component and flowable component are packaged separately for admixture at the point of care.

### VII. Methods of Use

The biphasic scaffolds of the present invention can be used in experimental modeling as well as therapeutic applications. Such experimental and therapeutic uses include, but are not limited to neovascularization, orthopedics, cardiovascular, neuronal, wound healing, hemostatic, drug screening and drug delivery, tissue regeneration, organoid, tissue (including soft tissue), and bone (re-)generation (e.g., spine, hip), dermatology, and dentistry (e.g., extraction sites and maxillofacial surgeries).

### EXAMPLES

### Example 1

### A Biphasic Cross Linked Carbohydrate Scaffold for Tissue Engineering

Background: HCCP is used clinically as an osteoconductive bone graft substitute (Koleva P M et al. Hyper-Crosslinked Carbohydrate Polymer for Repair of Critical-Sized Bone Defects. Biores Open Access. 2019 Jul. 1; 8(1):111-120.). Osteoconductive materials serve as scaffolding for new bone growth that is perpetuated by the native bone adjacent to a skeletal defect site. Osteoblasts from the bone defect margin infiltrate the graft framework and generate new bone that bridges the skeletal gap. Unlike osteoconductive materials, osteoinductive materials, such as bone morphogenetic protein-2 (BMP-2), are capable of stimulating osteoprogenitor cells to differentiate into cells of osteogenic lineage, which then begin forming the new bone matrix. Osteoinductive bone graft substitutes can expedite bone regeneration at defect sites and improve clinical outcomes in patients with compromised bone healing (Mckay W F, Peckham S M, Badura J M. A comprehensive clinical review of recombinant human bone morphogenetic protein-2 (INFUSE™ Bone Graft). Int Orthop. 2007 December; 31(6):729-34.). Despite efficacy in bone healing, collagen sponges infused with BMP-2 are associated with complications such as ectopic bone formation (Epstein N E. Complications due to the use of BMP/INFUSE in spine surgery: The evidence continues to mount. Surg Neurol Int. 2013 Jul. 9;4(Suppl 5): S343-52.) and anti-Bovine Collagen antibodies (18% of patients, according to INFUSE™ instructions for use), which may cause complications with repeat administration.

To investigate the ability of glucomannan-based scaffolds to act as a growth factor carrier, we created a biphasic HCCP delivery system composed of (1) porous HCCP scaffold chips in (2) a glucomannan gel. The HCCP scaffold chips provide long-term porosity and physical structure to facilitate tissue ingrowth. The transient glucomannan gel phase acts as a binding agent to hold the chips in place during surgical implantation, giving the biphasic HCCP system favorable handling characteristics like cohesion and moldability. Furthermore, the gel phase can be used as a slow-release delivery system when loaded with biologically active agents, such as growth factors, gene vectors, antibiotics, and chemotherapeutics.

Biphasic HCCP is prepared by mixing porous HCCP granules with glucomannan powder followed by hydration with saline, blood, bone marrow or other aqueous fluids. The ratio of liquid: powder (ml:g) determines the viscosity, from stiffer putty-like formulations around 10:1 or less, to well-hydrated but cohesive formulations around 20:1. Injectables can be made with higher liquid: powder ratios such as 40:1 or more. The ratio of solid phase HCCP chips: liquid: powder (g:ml:g) can be tailored to the specific requirements of the implant, with the ratio 1:30:2 yielding a moldable, cohesive, and well-hydrated implant.

Biologically active agents can be loaded in the biphasic HCCP by suspending them in the liquid used to hydrate the chips and glucomannan powder.

The osteoinductive potential of materials is typically evaluated in a heterotopic site in an animal model, where significantly less endogenous osteogenic factors are present to contribute to or enhance bone formation (reference ASTM f2529-13; Edwards J T, Diegmann M H, Scarborough N L. Osteoinduction of human demineralized bone: characterization in a rat model. Clinical Orthopaedics and Related Research. 1998December (357 ):219-228; Hartman E H M, Vehof J W M, de Ruijter J E, Spauwen P H M, Jansen J A. Ectopic bone formation in rats: the importance of vascularity of the acceptor site. Biomaterials 2004 December; 25(27):5831-7; Kirk J F, Ritter G, Waters C, Narisawa S, Millán J L, Talton J D. Osteoconductivity and osteoinductivity of NanoFUSE(®) DBM. Cell Tissue Bank. 2013 March; 14(1):33-44; Scott M A, Levi B, Askarinam A, Nguyen A, Rackohn T, Ting K, Soo C, James A W. Brief review of models of ectopic bone formation. Stem Cells Dev. 2012 Mar. 20;21(5):655-67; Lee H-R, Kim H-J, Ko J-S, Choi Y-S, Ahn M-W, Kim S, et al. (2013) Comparative Characteristics of Porous Bioceramics for an Osteogenic Response In Vitro and In Vivo. PLOS ONE 8(12): e84272; Asatrian, G., Chang, L., James, A.W. (2014). Muscle Pouch Implantation: An Ectopic Bone Formation Model. In: Christ, B., Oerlecke, J., Stock, P. (eds) Animal Models for Stem Cell Therapy. Methods in Molecular Biology, vol 1213. Humana Press, New York, NY.). We show here that biphasic HCCP can be loaded with recombinant BMP-2, yielding an osteoinductive bone graft substitute.

Study Design: The bone-forming capacity of each osteoinductive combination was evaluated using rodent intermuscular implants, a method widely accepted for studies of ectopic bone formation. (Scott et al., 2012, Kirk J F, et al., 2013; Lee H R, et al., 2013; Asatrian G, et al., 2014). Test articles (Biphasic HCCP+75 ul rhBMP-2) or control articles (INFUSE™ ACS+75 ul rhBMP-2) were implanted in the intermuscular pouch of the hindlimbs of rats. Each animal received 2 implants, one per hindlimb, for a total of 3 implants per treatment group. At approximately 28 days post-operatively, animals were sacrificed in a CO2 chamber. A necropsy was performed, and implants collected for histological processing and staining with H&E, Masson's Trichrome, and Alcian Blue. Osteoinduction, osteogenic changes, and bone tissue formation were evaluated microscopically.

### Implant Preparation

Biphasic HCCP+75 ul rhBMP-2: HCCP chips (10 mg) and konjac glucomannan powder (20 mg) were weighed in 2 mL microcentrifuge tubes and sterilized by autoclave. rhBMP-2 was reconstituted with SWFI according to package Instructions For Use (INFUSE™, Medtronic #7510100). Sterile rhBMP-2 solution (300 μL) was added to each microcentrifuge tube containing HCCP and konjac glucomannan powder and mixed approximately 1-3 minutes until a moldable paste-like composition formed. Using sterile technique, the biphasic HCCP was placed on a petri dish and cut into 4 equal pieces of approximately 7 mm×5 mm×5 mm dimensions. Each piece was used as one implant composed of 2.5 mg HCCP, 5 mg konjac glucomannan powder and 75 μL rhBMP-2 solution.

INFUSE™ ACS+75 μg rhBMP-2: Absorbable collagen sponge (ACS, INFUSE™, Medtronic, small kit, 1 inch×2 inch) was cut with a sharp scalpel to generate individual pieces in size proportional to the amount of BMP-2 solution added (approximately 10.9 mm×6.3 mm). Each piece of collagen sponge was then hydrated with 75 μL rhBMP-2 and incubated for at least 15 min at room temperature prior to implantation.

Animal Model: Female SAS Sprague Dawley rats were acquired at approximately 12 weeks of age (Charles River, Burlington, MA). Animals were acclimated for at least 7 days prior to initiating the study and were provided with unlimited water and standard pelleted laboratory rodent feed ad libitum.

Preoperative Procedure: A baseline body condition exam was performed preoperatively to confirm normal health status of all animals. Body weights were recorded 24 hours prior to surgery. At implant, animals were sedated using a ketamine (75 mg/kg SC) and midazolam (2 mg/kg SC) combination. One dose of extended-release buprenorphine XR (0.65 mg/kg) was administered subcutaneously for analgesia lasting up to 72 hours. Non-steroidal anti-inflammatory medications (NSAIDs) were not used due to potential confounding effects on the test system (see ASTM f2529-13). The hindquarters were shaved using clippers, and depilatory cream applied using a 5-min contact duration to remove all fur from the incision sites. Surgical plane anesthesia was induced and maintained using isoflurane (up to 3% for induction, 0.5-3% for maintenance) volatilized with oxygen (2 L/min) delivered through a precision vaporizer. Heart rate, SpO2, respiratory rate, and body temperature were monitored and recorded every 10 minutes during anesthesia. The surgery site was scrubbed with gauze saturated in chlorhexidine gluconate solution using a circular motion starting from the center of the surgery site and moving toward the periphery. Final sterile preparation of the surgical site was completed with chlorhexidine scrub prior to the first incision. Ophthalmic ointment was applied topically to the conjunctiva of each eye and may be reapplied throughout the procedure as needed.

Surgical Approach: A 1-2 cm skin incision was made in the hindlimb located superficial to the biceps femoris muscle. After the fascia were separated, the parallel muscle fiber groups of the biceps femoris muscle were located and separated by blunt dissection to create an intermuscular pouch. Care was taken to leave fiber groups intact and minimize bleeding. The assigned implant material was carefully inserted into the intermuscular pouch, ensuring that BMP-2 transfer to surrounding tissues was prevented. The muscle pouch, fascia, and skin incisions were closed at each level using 6-0 absorbable polyglactin suture in a simple interrupted pattern. Surgical skin adhesive was applied between skin sutures to reinforce closure. After incision closure, isoflurane was discontinued, and oxygen delivery continued until the animal regained consciousness.

Postoperative Procedure: Heart rate, SpO2, respiratory rate, and body temperature were monitored and recorded every 10 minutes during recovery. Once animals were mobile and displaying sternal recumbency, animals were returned to primary housing. Enrofloxacin (up to 5 mg/kg PO) was administered daily for 5 days postoperatively. Routine clinical observations were performed daily for 7 days postoperatively. Clinical observations included: general signs of well-being, feeding and drinking behavior, activity level, locomotion, wound closure, infection, and signs of pain and distress. Any evidence of adverse events was recorded in individual medical records.

Euthanasia and Tissue Collection: At approximately 28 days post-implantation, animals were euthanized in a CO2 chamber and necropsy performed. General integrity of the incision sites and any evidence of macroscopic reaction of the muscle tissue to the implant materials was noted. Implants were fixed in 10% neutral buffered formalin for approximately 24 hours and transferred to 70% ethanol. Specimens were processed, embedded in paraffin, sectioned, and stained with H&E and Masson's Trichrome at the University of California Medical Center. Alcian blue staining was performed by first deparaffinizing sections in xylene followed by rehydration in graded ethanols. Sections were incubated in the alcian blue solution for 30 minutes, then rinsed in running tap water for 5 minutes. Nuclei were visualized by counterstaining in nuclear fast red for 10 mins. Sections were dehydrated in graded ethanols before a coverslip was mounted.

Tissue Analysis: Osteoinduction was evaluated according to ASTM f2529-13 Standard Guide for in vivo Evaluation of Osteoinductive Potential for Materials Containing Demineralized Bone (DBM). Tissue sections were imaged with a dissection scope mounted with a Nikon D60 camera for whole tissue section images. A Nikon microscope with Moticam camera was used to image sections at 4× and 10× for histomorphometry. Two stained sections at 2 different levels (from ~30%, 50%, or 70% of the tissue block) from each of 3 replicate implants per group was selected for histomorphological analysis. Images were overlayed with a 10×10 grid in Image J to estimate the percentage of mature bone (H&E images at 4× magnification, 1 image per section) or cartilage (Alcian blue images at 10× magnification, 4 images per section) within the tissue area. Briefly, a count of all grid points overlying the entire section was compared with the grid points overlying bone or cartilage to determine the percentage composition of each tissue. Total tissue area was calculated in Image J using H&E images. Total bone or total cartilage area was determined by multiplying the total tissue area by the percentage of each tissue type. Differences between groups were determined by Student's t-test with significance set at p-values≤0.05.

### Results

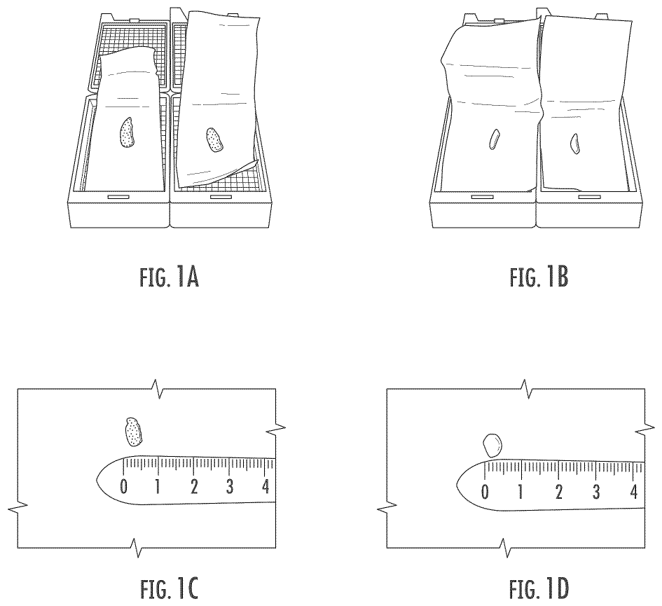

Macroscopic Appearance: No adverse reactions to implants were noted. Biphasic HCCP+rhBMP-2 Implant specimens collected at necropsy tended to show greater integration in surrounding tissue, were larger in volume (7 mm long×5 mm wide×4 mm deep), than ACS+rhBMP-2 implants (5 mm long×1.5 mm wide). Biphasic/BMP-2 implants exhibited palpably stiff areas of bone with a high degree of vascularization, evidenced by a deep red color. ACS/BMP-2 implants exhibited a hard shell, but appeared hollow inside and oozed a thick, yellow gel after bisection. See FIGS. 1A-D for representative images.

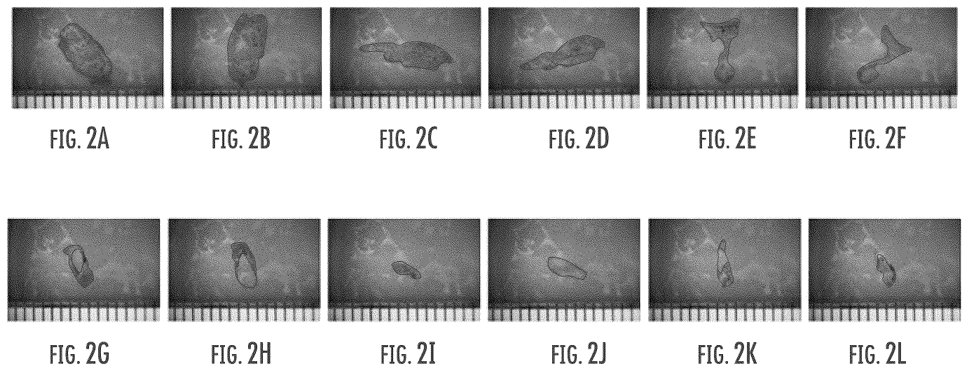

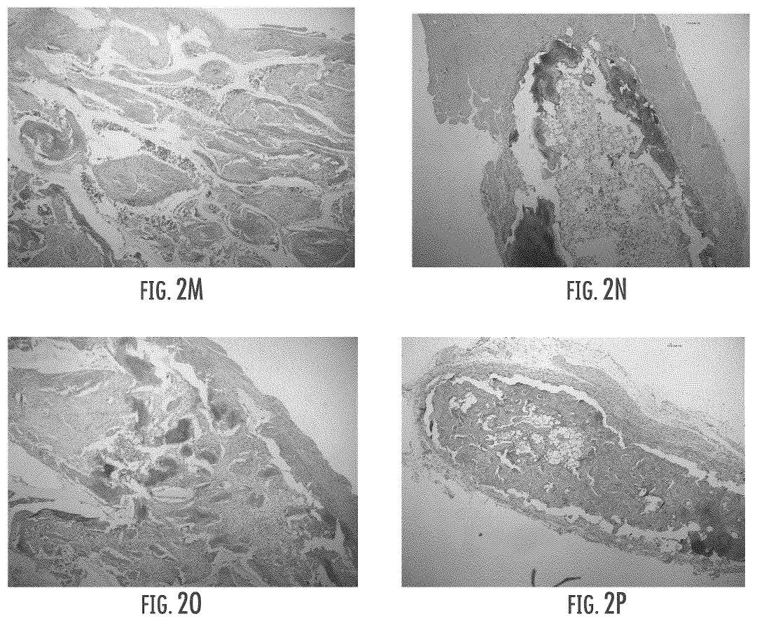

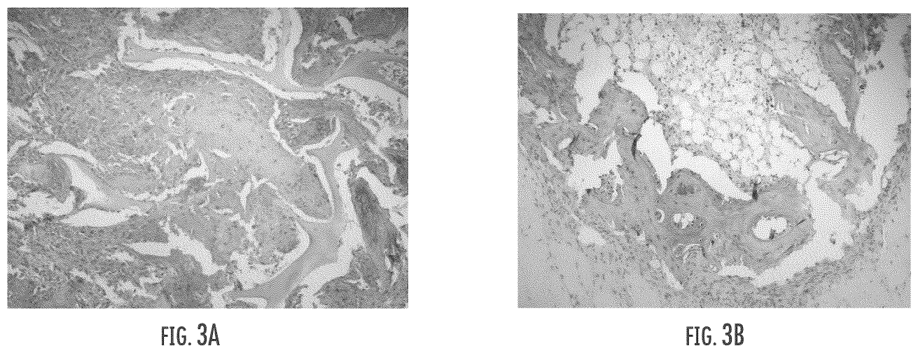

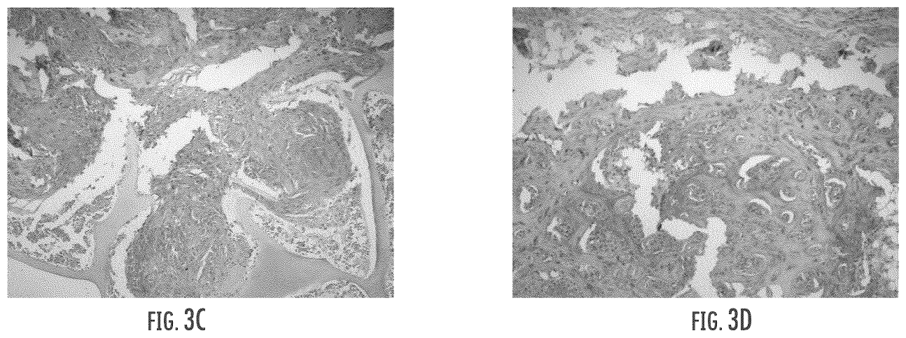

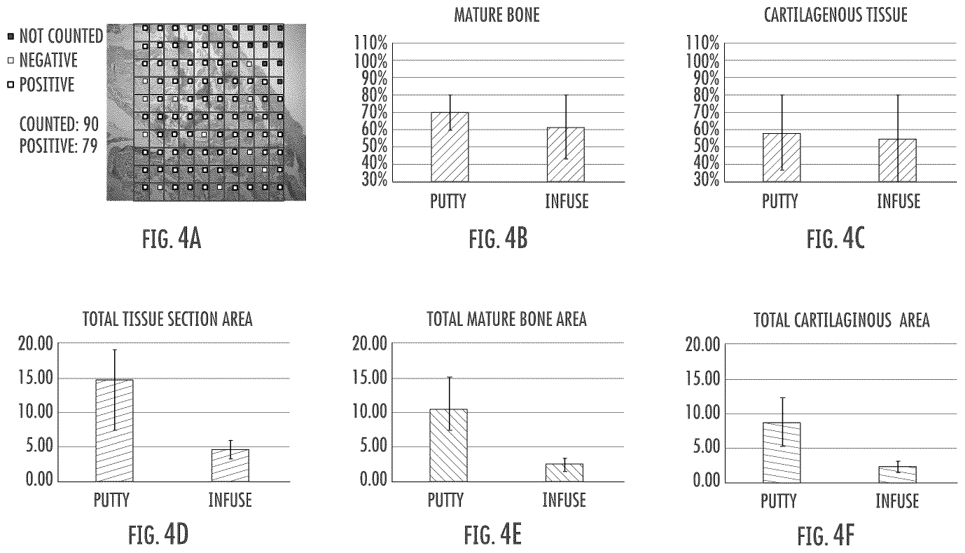

Microscopic Evaluation: H&E-stained sections were utilized to evaluate presence of chondrocytes, progenitor cells, and bone formation (see FIGS. 2A-L). Similar to macroscopic observations, the volume of tissue was greater in the HCCP implants. The presence of mature bone and cartilage was evaluated by Alcian blue staining (FIG. 3A-D). Alcian blue-stained sections were scored for mature bone or cartilagenous tissue as a percentage of total tissue area (FIG. 4A). Although the ratio of mature bone or cartilage to total tissue was similar (p=0.18) between biphasic HCCP+rhBMP-2 and ACS+rhBMP-2 implants (FIGS. 4B and 4C, respectively), the total tissue section area (FIG. 4D, p<0.001) was greater in biphasic HCCP+rhBMP-2 implants. The total mature bone area present in biphasic HCCP implants was increased 3.8-fold over the bone area present in INFUSE™ ACS implants (FIG. 4E, p<0.01). Total cartilaginous area in biphasic HCCP implants was increased 4.6-fold over INFUSE™ ACS implants (FIG. 4F, p<0.001).

### Conclusions

Biphasic HCCP implants with rhBMP-2 were compared with ACS+rhBMP-2 implants, the current standard of care for induction of bone in spinal fusion surgery, in a rat intermuscular model of osteoinduction. At 4 weeks post implant, the biphasic HCCP+rhBMP-2 implants were shown to be greater in volume and with a more robust and viable tissue morphology than the ACS+rhBMP-2 implants. Endochondral bone formation was significantly increased in biphasic HCCP+rhBMP-2 implants. In addition, cartilage precursor tissue and mature cartilage was nearly 5-fold greater in biphasic HCCP+rhBMP-2 implants as compared with the current standard of care.

Although the foregoing invention has been described in some detail by way of illustration and example for purposes of clarity of understanding, one of skill in the art will appreciate that certain changes and modifications may be practiced within the scope of the appended claims. Although particular features have been described herein with respect to certain embodiments, such features may be applied to any embodiment of the present invention. In addition, each reference provided herein is incorporated by reference in its entirety to the same extent as if each reference was individually incorporated by reference.

What is claimed is:

1. A biphasic scaffold comprising:
a. a solid porous component,
i. wherein the solid porous component comprises at least 50% (w/w) glucomannan, and
ii. wherein the solid porous component exhibits interconnected pores;
b. a flowable component disposed within the interconnected pores of the solid porous component,
i. wherein the flowable component comprises glucomannan powder and an aqueous fluid.

2. The biphasic scaffold of claim 1, wherein the ratio of aqueous fluid to glucomannan powder (ml: g) in the flowable component is 10:1 to 40:1.

3. The biphasic scaffold of claim 2, wherein the ratio of aqueous fluid to glucomannan powder (ml:g) in the flowable component is 10:1 to 20:1.

4. The biphasic scaffold of claim 1, wherein the ratio of aqueous fluid to glucomannan powder (ml:g) in the flowable component is at least 20:1.

5. The biphasic scaffold of claim 1, wherein the ratio of the solid porous component to the aqueous fluid to the glucomannan powder (g:ml:g) is about 1:30:2.

6. The biphasic scaffold of claim 1, further comprising at least one pharmaceutically active agent.

7. The biphasic scaffold of claim 6, wherein the pharmaceutically active agent is an antibiotic.

8. The biphasic scaffold of claim 6, wherein the pharmaceutically active agent is a chemotherapeutic agent.

9. The biphasic scaffold of claim 6, further comprising at least one tissue specific growth factor.

10. The biphasic scaffold of claim 1, further comprising at least one chemotactic agent.

11. The biphasic scaffold of claim 1, further comprising rhBMP2.

12. A method of treating a subject in need of bone repair by administering the biphasic scaffold of claim 11 to a bone injury site.

13. The method of treatment of claim 12, wherein the site is a spinal site.

14. The method of treatment of claim 12, wherein the site is a dental or maxillofacial site.
